(19) United States
(12) Patent Application Publication
(10) Pub. No.: US 2006/0154262 A1
(43) Pub. Date: Jul. 13, 2006

(54) Identification of genes involved in metastatic progression of cancer cells

(76) Inventors: Paul B. Fisher, Scarsdale, NY (US); Habib Boukerche, New York, NY (US)

Correspondence Address: BAKER & BOTTS, 30 ROCKEFELLER PLAZA, 44TH FLOOR, NEW YORK, NY, 10112, US

(21) Appl. No.: 11/032,766

(22) Filed: Jan. 11, 2005

(51) Int. Cl. C12Q 1/68; G01N 33/574; C07H 21/04; C12P 21/06; A61K 48/00

(52) U.S. Cl. 435/6; 435/7.23; 435/69.1; 435/320.1; 435/325; 530/350; 536/23.5; 514/44

(57) ABSTRACT

The present invention relates to the discovery, identification and characterization of an eight genes that are differentially expressed as a consequence of metastatic progression in human melanoma cells. Six of the identified metastasis elevated genes (MEG genes), encode for known proteins (MEG-1 through MEG-6). However, two of the identified genes, referred to herein as MEG-7 and MEG-8, represent novel genes. The present invention relates to methods for inhibiting the metastatic potential of cancer cells through inhibition of the MEG genes/gene products, as well as diagnostic methods for determining the metastatic potential of cells. The invention further relates to novel MEG-7 and MEG-8 nucleotides, host cell expression systems, MEG-7 and MEG-8 proteins, fusion proteins, and antibodies to the MEG-7 and MEG-8. The present invention also relates to the discovery that inhibition of the mda-9 gene (also referred to as syntenin), a gene found to be upregulated in metastatic cancer cells, results in a decrease in the invasive and migratory properties of such cells. Thus, the present invention relates also to methods and compositions for inhibiting mda-9/syntenin activity.

### GRANT INFORMATION

The invention disclosed herein was made with United States Government support under National Institutes of Health Grant CA35675, CA97318 and A98712 from the U.S. Department of Health and Human Services. Accordingly, the U.S. Government may have certain rights herein.

### 1. INTRODUCTION

The present invention relates to the discovery, identification and characterization of a eight genes that are differentially expressed as a consequence of metastatic progression in human melanoma cells. Six of the identified metastasis elevated genes (MEG genes) encode known proteins (MEG-1 through MEG-6). However, two of the identified genes, referred to herein as MEG-7 and MEG-8, represent novel genes. The present invention relates to methods for inhibiting the metastatic potential of cancer cells through inhibition of the MEG genes/gene products, as well as diagnostic methods for determining the metastatic potential of cells. The invention further relates to novel MEG-7 and MEG-8 nucleotides, host cell expression systems, MEG-7 and MEG-8 proteins, fusion proteins, and antibodies to MEG-7 and MEG-8.

The present invention also relates to the discovery that inhibition of the mda-9 gene (also referred to as syntenin), a gene found to be upregulated in metastatic cancer cells, results in a decrease in the invasive and migratory properties of such cells. Thus, the present invention relates also to methods and compositions for inhibiting mda-9/syntenin activity.

### 2. BACKGROUND OF THE INVENTION

### 2.1. Metastatic Cancer

Malignant melanoma, a tumor originating from melanocytes, is among the most aggressive human cancers, the incidence of which in recent years has been steadily increasing at a rate that exceeds all other malignant neoplasms. At present, the incidence increases by 6 to 7% yearly, doubling the population risk every 10 years since 1950 (Bevona et al., 2002, Dermatol. Clin. 20:589-595). In 2004, the American Cancer Society estimates that there will be 55,100 new cases of melanoma and 7,910 people will die of this disease. Localized melanoma lesions can generally be removed by surgical excision resulting in a high cure rate, with a 5-year survival rate of more than 80%. However, the prognosis of melanoma patients with lymphatic and distant metastases becomes progressively worse with an estimated median survival time of 8 months and a 5-year survival rate of less than 5%. Moreover, in spite of significant improvements in surgical, local and systemic therapy, curative therapy is not available for these patients, and the vast majority will succumb to disease progression (Eigentler et al., 2003, Lancet Oncol. 4:748-759).

Most melanoma lesions are believed to develop from melanocytes along a continuum of progressive stages. During the period of radial growth phase, malignant cells are confined to the epidermis and are not associated with metastasis (Bogenrieder and Herlyn, 2002, Crit. Rev. Oncol. Hematol. 44:1-15). Eventually, the melanoma evolves into the vertical growth phase and the malignant cell population invades the dermis and the potential for cells to metastasize commences. As the tumor cells clinically progress to metastatic cancer, the development of tumor heterogeneity within the primary tumor, mainly driven by genomic instability, generates aggressive subpopulations of tumor cells with increased capacity to escape from immune surveillance and local growth control mechanisms, invade through the basement membrane, intravasate into blood vessels and/or the lymphatic system, and finally extravasate and grow at the target organ (Fisher, 1984, Tumor Promotion and Cocarcinogenesis In Vitro, Mechanisms of Tumor Promotion, pp. 57-123; Fidler 2002, Semin. Cancer Biol. 12:89-96). Based on the complexity and multitude of cellular interactions involved in the metastatic cascade, it is not surprising that a number of proteins have been associated with tumor cell dissemination, including transcription factors, growth factors, signaling proteins, chemokines, extracellular proteases (matrix metalloproteinases), motility factors and adhesion molecules (Bar-Eli, 2002, Cancer Biol. Ther. 1:459-465; Bogenrieder and Herlyn, 2002, Crit. Rev. Oncol. Hematol. 44:1-15).

Over the past several years, hybridoma technology has enabled the creation of monoclonal antibodies which recognize cell surface antigens with a preferential expression for one or few stages of this process (Leon et al., 1994, Pharmacol. Ther. 61:237-278). Many of these antibodies have helped in the identification of a number of cell surface adhesion molecules, such as integrins, ICAM-1/CD54, CD44, and MUC18/CD146 that have been implicated in both cell-substratum and cell-cell adhesion (Bar-Eli, 2002, Cancer Biol. Ther. 1:459-465). In addition to this classical approach to antibody production, several research groups have developed subtracted immunological approaches to improve the response to weak and rare antigens (Williams et al., 1992, Biotechniques 12:842-847; Shen et al., 1994, J. Natl. Cancer Inst. 86:91-98; Fisher, 1995, Pharmaceutical Tech. 19 (9):42-48). This has led to the identification of novel monoclonal antibodies directed against unique epitopes present on one cell type and not another which was otherwise unobtainable by traditional hybridoma technology (Shen et al., 1994, J. Natl. Cancer Inst. 86:91-98; Shen et al., 1996, Proc. Natl. Acad. Sci. U.S.A. 93:7252-7257; Wulf et al., 1996 J. Cancer Res. Clin. Oncol. 122:476-482; Boukerche et al., 2000, Cancer Res. 60:5848-5856).

An orthotopic model of tumor cell metastasis comprising several variants selected in vivo from a poorly metastatic human parental melanoma cell line for their ability to give rise to multiple spontaneous metastases in the lungs has been developed (Boukerche et al., 1994, Eur. J. Biochem. 220:485-491). When these variants were subcutaneously grafted in immunosuppressed newborn rats, they exhibited a wide spectrum in their respective metastatic incidence, ranging from near absence to 100% lung nodule formation. This represents an ideal model for defining the biochemical and molecular basis of metastatic progression in melanoma since metastatic incidence was stable after continuous growth in monolayer culture and a characteristic trait of each cell line.

As one approach to elucidate the mechanism(s) by which nonmetastatic cells acquire metastatic capacity, a subtractive immunological strategy was used to raise monoclonal antibodies to cell surface antigens that showed minimal expression on the parental melanoma cell line but were significantly enhanced in expression on the metastatic melanoma variants (Boukerche et al., 2000, Cancer Res. 60:5848-5856). These studies led to the recognition of an unidentified cell-cell adhesion molecule of 55-kDa which is specifically up-regulated in primary melanoma, namely during the VGP and in metastatic lesions (Boukerche et al., 2000, supra; Baril et al., 2002, Int. J. Cancer 99:315-22). Appearance of such a phenotypic marker of primary melanomas undergoing the radial to VGP transition strongly supports the concept that this experimental model mimics the early events of metastasis in humans. Multiple molecular biological strategies are now available that allow global identification of genes whose expression profiles differ between two specific physiological settings or tissues, including differential RNA display, subtraction hybridization, reciprocal subtraction differential RNA display (RSDD), representational difference analysis (RDA), serial analysis of gene expression (SAGE), and cDNA microarray hybridization (Liang and Pardee, 1992, Science 257:967-971; Jiang and Fisher, 1993, Mol. Cell. Different. 1:285-299; Lisitsyn et al., 1993, Science 259:946-951; Velculescu et al., 1995, Science 270:484-487; Schena et al., 1995, Science 270:467-470; Kang et al., 1998, Proc. Natl. Acad. Sci. U.S.A. 95:13788-13793; Shimkets et al., 1999, Nat. Biotechnol. 17:798-803; Weeraratna et al., 2004, Oncogene 18:2264-2274). Although differential and subtraction hybridization screening have proven valuable in many applications, they are not trouble-free and differentially expressed clones representing rare mRNAs frequently escape detection (Bittner et al, 2000, Nature 406:536-540). In addition, the development of tumor heterogeneity within the primary tumor and metastatic lesions adds a further level of complexity in identifying those metastasis-regulatory genes that are mandatory for metastasis formation. Indeed, several genes differentially expressed in malignant melanoma that had not been previously described are still being identified through the use of these methods (Mitchell et al., 2000, Cancer Res. 60:6448-6456; Deichmann et al, 2001, Melanoma Res. 11:577- 585; Zendman et al., 2002, Int. J. Cancer 195-204; Goldberg et al., 2003, Cancer Res. 63:432-440; Weeraratna et al., 2004, Oncogene 18:2264-2274). These studies, however, have focused primarily on late rather than early steps of neoplastic progression of melanocytic cells.

### 2.2. Known MEG Genes

ENDRB (“MEG-1”) was originally isolated from cultured porcine aortic endothelial cells as a G-protein-coupled heptahelical receptor that binds all endothelin isopeptides (ET-1, ET-2, ET-3) with equal affinity. The portion shares high sequence homology with another receptor known as ENDRA that binds selectively to ET-1 and ET-2. In view of the mitogenic effects of the Ets for various cell types in vitro and in vivo, the ENDRB receptor has been extensively studied in several cancers including melanoma (Yohn et al., 1994, Biochem. Biophys. Res. Commun. 30:449-457; Nelson et al., 2003, Nat Rev. Cancer 3:110-116; Bittner et al., 2000, Nature 406:536-540). The physiological role of ENDRB in melanocyte physiology was demonstrated by the fact that ET3 enhances the proliferation and delays the differentiation of melanocyte precursors, an effect correlated with upregulation of ENDRB (Nelson et al., 2003, supra). Similarly, human melanocyte precursors fail to develop in individuals with mutations in the gene encoding ENDTRB, a syndrome known as Hirschsprung's disease characterized by pigmentation abnormalities (Puffenberger et al., 1994, Cell 79:257-1266). While initial studies found decreased expression levels in metastatic melanoma cell lines (Eberle et al., 1999, J. Invest. Dermatol. 112:925-932), a recent report documented gradual increases in ENDRB expression on both the protein and RNA level from common nevus to metastatic malignant melanoma (Demunter et al., 2001, Virchows Arch. 438:485-491). Moreover, it was recently shown that a selective ENDRB agonist such as BQ788 significantly inhibited tumor growth in vivo by enhancing melanoma cell death (Bagnato et al., 2004, Cancer Res. 64:1436-1443).

The non-integrin receptor, known as the 67-kDa laminin receptor (“MEG-2”) has been previously shown to be a high affinity receptor for laminin, a major component of the ECM (extracellular matrix) including basement membranes through which tumor cells adhere, migrate, proliferate and grow in host tissue (Fulop and Larbi, 2002, Semin. Cancer Biol. 12:219-229). Cell surface expression of the 67LR was shown, in several in vitro and in vivo systems, to be dramatically increased in highly metastatic cells including colorectal, gastric, ovary, cervical, breast and non-small cell lung carcinomas, and lymphomas (Fulop and Larbi, 2002, Semin. Cancer Biol. 12:219-229). Despite being one of the best characterized members of the laminin receptor with a number of potential functions, the precise structure of this molecule has not been elucidated, and only the cDNA encoding a cytoplasmic precursor of 37-kDa (37LRP) has been identified which shows intriguing identity to the ribosomal-associated protein p40 (Rao et al., 1989, Biochemistry 28:7476-7486). Although these initial studies suggest that the 37LRP/P40 protein is a component of the translational machinery involved in protein synthesis, this laminin precursor protein was recently recognized as a prototype of evolutionary molecules that acquire laminin-binding ability during evolution through the presence of a 20 amino-acid peptide (Ardini et al., 1998, Mol. Biol. Evol. 15:1017-1125). These data are consistent with the observations that a posttranslational modification of the 37LRP, involving acylation, by the fatty acids palmitate, and stearate, leads to dimerization. Interestingly, the mature membrane form of the 67-kDa laminin receptor has been shown to physically associate with integrin during laminin recognition (Ardini et al., 1997, J. Biol. Chem. 272:2342-2345). Previous work indicates that the metastatic variants described in this report did not differ from the parental cell line M4Beu in their respective integrin subunits profile including α1, α2, α3, α4, α5, α6, αv and β1 (Boukerche et al., 1994, Eur. J. Biochem. 220:485-491; Boukerche et al., 2000, Cancer Res. 60:5848-5856).

The Ku antigen (“MEG-3”) has been shown to be essential for DNA repair of double-strand breaks (Tuteja et al., 2000, Crit. Rev. Biochem. Mol. Biol. 35:1-33). It was originally identified as a non-histone binding DNA protein recognized by sera from Japanese patients with scleroderma polyomitosis overlap syndrome (Mimori et al., 1990, Proc. Natl. Acad. Sci. U.S.A. 87:1777-1781). It consists of a heterodimeric protein of Ku7O and Ku80 subunits that bind with high affinity to altered DNA, including double-stranded DNA breaks (DSBs) (Tuteja et al., 2000, Crit. Rev. Biochem. Mol. Biol. 35:1-33). Such binding is followed by the recruitment of the catalytic subunit of the 460-kDa protein DNA-dependant protein kinase catalytic subunit leading to phosphorylation of DNA-bound protein (Tuteja et al., 2000, supra). These data indicate that Ku antigen may function as a ‘caretaker’ gene involved in monitoring the stability of the genome and suppressing chromosomal translocation.

With respect to Ku antigens, recent studies have shown that functional dysregulation of Ku70 and Ku80 seems to be of greater biological importance than the loss of expression in metastatic tumor spread (Korabiowska et al., 2002, Mod. Pathol. 15:426-433). The mechanism of this dysregulation is not known but might be due to a defect in heterodimerization of Ku proteins which has been shown to have physiological importance for efficient nuclear translocation of each Ku (Koike et al., 2001, J. Biol. Chem. 276:11167-11173). It has been shown that Ku80 is overexpressed on the cell surface on a variety of tumor cells, including leukemia and solid tumor cell lines, and mediates both homotypic and heterotypic cell adhesion to fibronectin (Tai et al., 2002, supra).

IRAK-1 (“MEG-4”) is a serine/threonine kinase associated with the interleukin-1 receptor (Cao et al., 1996, Science 271:1128-1131). Although IRAK-1 was originally identified as a signal transducer for the proinflammatory cytokine interleukin-1 (IL-1), it is now recognized as a central serine/threonine kinase player of physiological importance in signal transduction of other members of the Toll-like receptor (TLR)/IL-1 (IL-1R) family including IL-1R, IL-R18R, ST2/T1 and the TLR-1 and 10 (Janssens and Beyaert, 2003, Mol. Cell 11:293-302). The IRAK family is currently known to contain four related kinases including two splice variants termed IRAK-1b that lacks only 30 amino acids at the C-terminal end of the kinase domain and IRAK-1s which is more truncated (Janssens and Beyaert, 2003, supra).

L37a (“MEG-5”) is a ribosomal protein (Barnard et al., 1994, Biochim. Biophys. Acta 1218:425-428). Over-expression of several ribosomal proteins have been reported in a variety of cancers, including prostate, colon, liver, and breast carcinomas which might reflect the higher level of metabolic activity as compared to melanocytes. It is of interest regarding L37a that it contains several putative serine/threonine phosphorylation sites, suggesting that it might be subject to regulatory cascade of kinases and phosphatases (Barnard et al., 1994, supra).

The Na⁺/K⁺-ATPase alpha subunit (“MEG-6”), also known as the sodium pump (Xie and Cai, 2003, Mol. Interv. 3:157-168), consists of three subunits, the α-subunit is the catalytic-subunit while the β- and the γ-subunits have modulatory roles in Na⁺/K⁺-ATPase activity. A recent study demonstrated the physiological importance of Na⁺/K⁺-ATPase activity in the assembly of junctional complexes and polarity of epithelial cells through the regulation of the MAPK signaling pathway (Rajasekaran et al., 2001, Mol. Biol. Cell. 12:3717-3732). It has been demonstrated that B16 melanoma variants selected for growth inhibition in vivo by dimethylthiourea have a decreased Na⁺/K⁺-ATPase activity (Fux et al., 1991, Br. J. Cancer 63:489-494).

### 3. SUMMARY OF THE INVENTION

The present invention relates to the discovery, identification and characterization of a group of genes that have been found to be differentially expressed as a consequence of metastatic progression in human melanoma cells. As demonstrated by Northern Blot analysis, expression of the MEG-1 through MEG-8 transcripts is upregulated in highly metastatic cell lines.

The present invention further relates to methods and compositions for inhibiting the metastatic potential of cells comprising administering to a subject afflicted with cancer an inhibitor of one or more of the MEG genes described herein. Such inhibitors include, for example, antisense molecules as well as interfering RNA (“iRNA, RNAi or siRNA”) molecules.

The present invention still further relates to diagnostic methods for determining the metastatic potential of cancer cells. Such diagnostic methods comprise assaying a tissue sample, for the level of MEG gene expression wherein detection of increased levels of expression as compared to expression in normal cells or tissue, correlate with an increase in the metastatic potential of cells.

Although MEG-1 through MEG-6 are known genes, the MEG-7 and MEG-8 are two newly identified genes. Thus, the present invention encompasses MEG-7 and MEG-8 nucleotides, host cells expressing such nucleotides and the expression products of such nucleotides. The invention encompasses MEG-7 and MEG-8 protein, MEG-7 and MEG-8 fusion proteins, and antibodies to the MEG-7 and MEG-8 proteins.

The present invention also relates to the discovery that inhibition of mda-9/syntenin, an additional gene found to be overexpressed in metastatic cancer cells, can decrease the invasive and migratory properties of cells. Thus, the present invention relates to methods and compositions for inhibiting the activity of mda-9. Such inhibitors include, for example, antisense and iRNA molecules.

### 4. BRIEF DESCRIPTION OF THE DRAWINGS

FIG. 1: Schematic representation of the RaSH approach as applied to a highly metastatic variant T1P26R selected in vivo after orthotopic inoculation in immunosuppressed newborn rats of the parental M4Beu melanoma cell line. This scheme involves construction of tester (T1P26R) and driver (M4Beu) melanoma cDNA libraries, followed by digestion of only the tester library with XhoI. After hybridization, differentially expressed sequences are cloned into XhoI-digested vectors, resulting in a subtracted cDNA library enriched for metastasis elevated genes (MEGs) displaying elevated expression in melanoma cells displaying an enhanced metastatic phenotype.

FIG. 2: Ethidium bromide staining of PCR-based cDNA libraries prepared from M4Beu and T1P26R melanoma cell lines. Poly(A)-RNA (1 μg) from M4Beu cells (driver) or T1P26R cells (tester) were used for first-strand and second-strand cDNA synthesis. After DpnII digestion and adaptation ligation, an aliquot of the mixture were used for PCR amplification. The PCR products were purified and portions of the tester PCR products were digested with XhoI. Aliquots (5 μl) of the two sets of cDNA libraries were separated on a 1% agarose gel and stained with ethidium bromide.

FIG. 3: Reverse Northern blot analysis of differentially expressed sequences identified by RaSH. Equal amounts of PCR amplified products (5 μl) from random bacterial clones of RaSH-derived libraries were loaded onto 1.2% agarose gels. Samples were electrophoresed and transferred onto nylon membranes. The membranes were then hybridized with ³²P-labeled cDNA reverse transcribed RNA samples from poorly metastatic parental M4Beu cell line (panels A, C, E) or highly metastatic variant T1P26R (panels B, D, F). Blots were exposed to autoradiography. Arrows indicate differentially expressed cDNA fragments in the T1P26R variant melanoma cell line compared with M4Beu parental melanoma cells.

FIG. 4: Confirmation of RaSH-selected cDNA clones (MEG-1 to MEG-6) representing authentic differentially expressed genes upregulated in highly metastatic cell lines by Northern blot analysis. Total RNA from the indicated immortal melanocyte (FM516-SV) or melanoma cell line were transferred to positively charged nylon membranes and probed with α³²P-dCTP differentially expressed MEG fragments identified by RaSH. Blots were stripped and subsequently probed with GAPDH.

FIG. 5: Confirmation of RaSH-selected cDNA clones (MEG-7 and MEG-8) representing authentic differentially expressed genes upregulated in highly metastatic cell lines by Northern blot analysis. Total RNA from immortal melanocytes (FM516-SV) or the indicated melanoma cell line were transferred to positively charged nylon membranes and probed with α³²P-dCTP differentially expressed MEG fragments identified by RaSH. Blots were stripped and subsequently probed with GAPDH.

FIG. 6: MEG-7 nucleotide sequence (SEQ. ID NO. 1).

FIG. 7: MEG-8 nucleotide sequence (SEQ. ID NO. 2).

FIG. 8A: Effect of inhibition of mda-9/syntenin on anchorage independent growth of Hela cells. Cells transfected with either empty vector pcDNA3.1 or plasmid expressing antisense mda-9/syntenin are analyzed for anchorage independent growth in soft agar.

FIG. 8B: Effect of inhibition of mda-9/syntenin on migration of Hela cells. Migration of cells transfected with either control scrambled siRNA or siRNA against mda-9/syntenin, through Matrigel® coated filters were analyzed.

### 5. DETAILED DESCRIPTION OF THE INVENTION

The present invention relates to the discovery, identification and characterization of genes, referred to as metastasis elevated genes (MEG genes), that have been found to be differentially expressed as a consequence of metastatic progression in human melanoma cells. The present invention further relates to methods and compositions for inhibiting the metastatic potential of cells comprising administering to a subject afflicted with cancer an inhibitor of one or more of the MEG genes described herein. The invention also relates to diagnostic and/or prognostic methods designed to determine the metastatic potential of cells.

Two of the identified genes, referred to herein as MEG-7 and MEG-8, represent novel genes. Thus, the present invention encompasses MEG-7 and MEG-8 nucleotides, MEG-7 and MEG-8 proteins and peptides as well as antibodies to the MEG-7 and MEG-8 protein. The invention also relates to antisense MEG-7 and MEG-8 molecules as well as MEG-7 and MEG-8 siRNA molecules.

In addition, the present invention relates to inhibition of mda-9/syntenin gene activity as a means for modulating the metastatic activity of cancer cells. This particular aspect of the invention is based on the observation that overexpression of mda-9/syntenin antisense or siRNA significantly decreased anchorage-independent growth in soft agar and migration of cells through Matrigel®-coated filters.

Various aspects of the invention are described in greater detail in the subsections below.

### 5.1. MEG-1 Through MEG-6 Genes

The present invention relates to the discovery, identification and characterization of eight genes that are differentially expressed as a consequence of metastatic progression in human melanoma cells. Six of the identified metastasis elevated genes (MEG genes) encode for known proteins.

MEG-1, which encodes endothelin receptor B (ENDRB), was detected as an mRNA transcript of ˜1.7 kb and was expressed in normal melanocytes and all of the melanoma cell lines. However, its expression level was greater (˜3.2- to ˜3.5-fold) in metastatic cells than in non-metastatic M4Beu melanoma cells (Eberle et al., 1995 Pigment Cell Res, 8: 307 313).

MEG-2 was detected as an mRNA transcript of ˜1 kb that was significantly up-regulated (˜2.5- to 3 fold) in metastatic versus nonmetastatic melanoma cells. The MEG-2 transcript showed strong homology with a non-integrin receptor, known as the 67-kDa laminin receptor (67LR) which had previously been shown to be a high affinity receptor for laminin. Laminin is a major component of the ECM (extracellular matrix) including basement membranes through which tumor cells adhere, migrate, proliferate and grow in host tissue (Grosso et al., 1991 Biochemistry. 30(13):3346-3350). Characterization of a putative clone for the 67-kilodalton elastin/laminin receptor suggests that it encodes a cytoplasmic protein rather than a cell surface receptor.

The MEG-3 gene, found to be upregulated in metastatic cells is Ku antigen which has been shown to be essential for DNA repair of double-strand breaks. Ku antigen was originally identified as a non-histone binding DNA protein recognized by sera from Japanese patients with scleroderma polyomitosis overlap syndrome. It is believed that Ku antigen may function as a ‘caretaker’ gene involved in monitoring the stability of the genome and suppressing chromosomal translocation (Tuteja et al., 2000, Crit. Rev. Biochem. Mol. Biol. 35:1-33)

MEG-4 encodes an mRNA transcript of ˜3.5-kb, which is dramatically upregulated in metastatic melanoma cells. This transcript is moderately up-regulated in the metastatic C8161 melanoma cell line, but its expression level was significantly enhanced by ˜3- to ˜3.5-fold in the metastatic variants 7GP122 and T1PP26R as compared to parental poorly metastatic melanoma cell line M4Beu. The MEG-4 cDNA clone displayed homology with a previously described serine/threonine kinase associated with the interleukin-1 receptor, known as IRAK-1. The data presented herein demonstrates for the first time up-regulation of the IRAK-1 gene in melanoma cells (Janssens and Beyaert, 2003, Mol. Cell 11:293-302).

MEG-5 which was detected as an mRNA transcript of ˜0.5 kb in length which is significantly up-regulated in all metastatic cells (˜3- to 4.2-fold). The MEG-5 cDNA clone showed homology to ribosomal protein L37a (Barnard et al., 1994, Biochim. Biophys. Acta 1218:425-428; Saha et al., 1993 Gene 132: 285-289,)

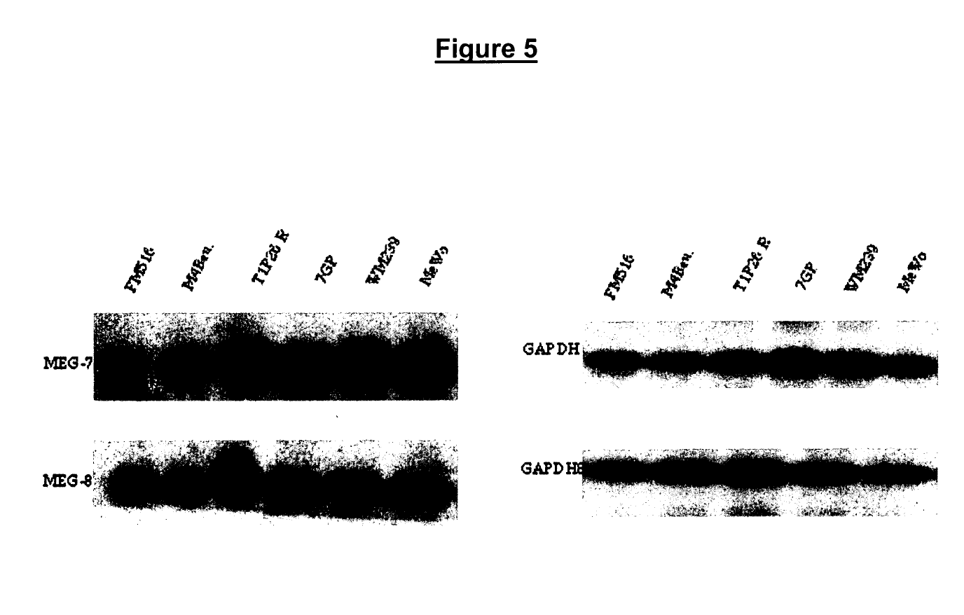

MEG-6, which was detected as an mRNA transcript of ˜2.5 kb, is homologous to Na⁺/K⁺-ATPase alpha subunit, also known as the sodium pump. The MEG-6 gene was significantly up-regulated in melanoma cells with high metastatic potential (˜3 to ˜4.3-fold) (FIG. 5). The data presented herein demonstrates for the first time an association between expression of Na⁺/K⁺-ATPase and melanocytic progression (Xie and Cai, 2003, Mol. Interv. 3:157-168).

### 5.2. The MEG-7 and MEG-8 Genes

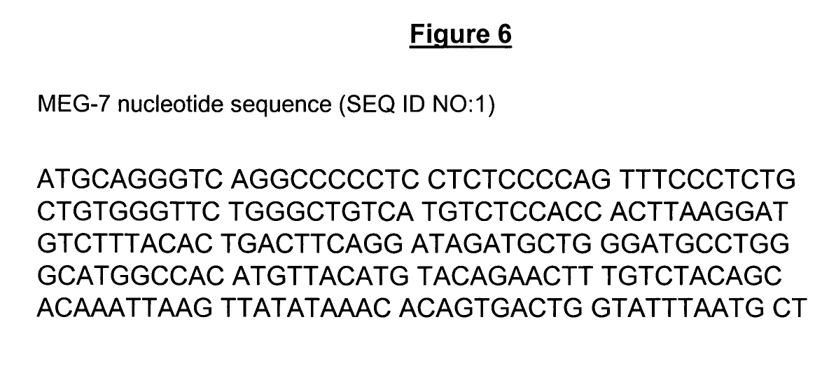

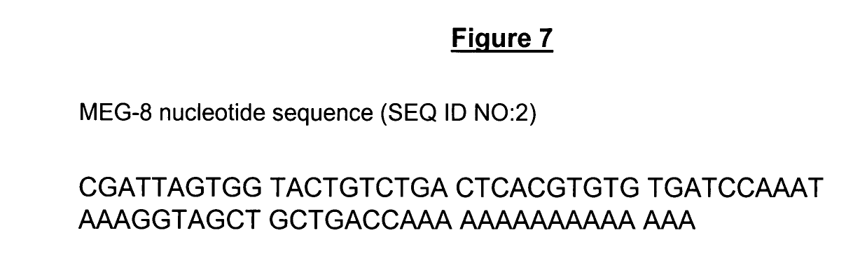

In addition to the MEG-1 through MEG-6 genes, two novel genes referred to as MEG-7 and MEG-8, were identified. A nucleotide sequence of the MEG-7 gene is shown in FIG. 6 (SEQ ID NO: 1). A nucleotide sequence of the MEG-8 gene is shown in FIG. 7 (SEQ ID NO:2). It is likely that both SEQ ID NO:1 and SEQ ID NO:2 are partial sequences of the MEG-7 and MEG-8 genes, respectively.

The MEG-7 nucleotide sequences of the invention include: (a) the DNA sequences shown in FIG. 6 (SEQ ID NO. 1); (b) any nucleotide sequence that (i) hybridizes to the nucleotide sequence set forth in (a) under stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F.M. et al., eds., 1989, Current Protocols in Molecular Biology, Vol. I, Green Publishing Associates, Inc., and John Wiley & sons, Inc., New York, at p. 2.10.3) and (ii) encodes a gene product that is elevated in metastatic cells; (c) any nucleotide sequence that hybridizes to a DNA sequence that encodes the MEG-7 amino acid sequence, under less stringent conditions, such as moderately stringent conditions, e.g., washing in 0.2×SSC/0.1% SDS at 42° C. (Ausubel et al., 1989 supra), yet which still encodes a functionally equivalent MEG-7 gene product; and (d) any nucleotide sequence which is at least 80, 85, 90 or 95 percent homologous to the nucleic acid sequence shown in FIG. 6 (SEQ ID NO. 1) or its complement, as determined by standard homology determining software, such as BLAST or FASTA. Functional equivalents of the MEG-7 protein include naturally occurring MEG-7 present in species other than humans. The invention also includes degenerate variants and complements of sequences (a) through (d). The invention also includes nucleic acid molecules that may encode or act as MEG-7 antisense molecules, useful, for example, in MEG-7 gene regulation (for and/or as antisense primers in amplification reactions of MEG-7 gene nucleic acid sequences). In addition, the invention includes nucleic acid molecules that may comprise of or encode inhibitory RNA molecules (RNAi) that have the capacity to specifically target and reduce or ablate levels MEG-7 mRNA and thereby MEG-7 protein expression. These MEG-7 specific RNAi molecules include synthetic double stranded RNA or may be derived from appropriate plasmid or viral expression vectors that express functional RNAi molecules that reduce expression of MEG-7 gene product.

The MEG-8 nucleotide sequences of the invention include: (a) the DNA sequences shown in FIG. 7 (SEQ ID NO. 2); (b) any nucleotide sequence that (i) hybridizes to the nucleotide sequence set forth in (a) under stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F.M. et al., eds., 1989, Current Protocols in Molecular Biology, Vol. I, Green Publishing Associates, Inc., and John Wiley & sons, Inc., New York, at p. 2.10.3) and (ii) encodes a gene product that is elevated in metastatic cells; (c) any nucleotide sequence that hybridizes to a DNA sequence that encodes the amino acid sequence of MEG-8, under less stringent conditions, such as moderately stringent conditions, e.g., washing in 0.2×SSC/0.1% SDS at 42° C. (Ausubel et al., 1989 supra), yet which still encodes a functionally equivalent MEG-8 gene product; and (d) any nucleotide sequence which is at least 80, 85, 90 or 95 percent homologous to the nucleic acid sequence shown in FIG. 7 (SEQ ID NO. 2) or its complement, as determined by standard homology determining software, such as BLAST or FASTA. Functional equivalents of the MEG-8 protein include naturally occurring MEG-8 present in species other than humans. The invention also includes degenerate variants and complements of sequences (a) through (d). The invention also includes nucleic acid molecules that may encode or act as MEG-8 antisense molecules, useful, for example, in MEG-8 gene regulation (for and/or as antisense primers in amplification reactions of MEG-8 gene nucleic acid sequences). In addition, the invention includes nucleic acid molecules that may comprise of or encode inhibitory RNA molecules (RNAi) that have the capacity to specifically target and reduce or ablate levels MEG-8 mRNA and thereby MEG-8 protein expression. These MEG-8 specific RNAi molecules include synthetic double stranded RNA or may be derived from appropriate plasmid or viral expression vectors that express functional RNAi molecules that reduce expression of MEG-8 gene product.

In addition to the MEG-7 and MEG-8 nucleotide sequences described above, homologs of the MEG-7 or MEG-8 gene present in other species can be identified and readily isolated, without undue experimentation, by molecular biological techniques well known in the art. For example, cDNA libraries, or genomic DNA libraries derived from the organism of interest can be screened by hybridization using the nucleotides described herein as hybridization or amplification probes.

The invention also encompasses nucleotide sequences that encode mutant MEG-7 or MEG-8, peptide fragments of MEG-7 or MEG-8, truncated MEG-7 or MEG-8, and MEG-7 or MEG-8 fusion proteins. Certain of these truncated or mutant proteins may act as dominant-negative inhibitors of the native MEG-7 or MEG-8 protein. Nucleotides encoding fusion proteins may include but are not limited to full length MEG-7 or MEG-8 protein, truncated MEG-7 or MEG-8, or peptide fragments of MEG-7 or MEG-8 fused to an unrelated protein or peptide such as an enzyme, fluorescent protein, luminescent protein, etc., which can be used as a marker.

Additionally, MEG-7 and MEG-8 nucleotide sequences may be isolated using a variety of different methods known to those skilled in the art. For example, a cDNA library constructed using RNA from cells known to express MEG-7 or MEG-8 can be screened using a labeled MEG-7 or MEG-8 probe. Alternatively, a genomic library may be screened to derive nucleic acid molecules encoding the MEG-7 or MEG-8 protein. Further, MEG-7 or MEG-8 nucleic acid sequences may be derived by performing PCR using two oligonucleotide primers designed on the basis of the MEG-7 or MEG-8 nucleotide sequences disclosed herein. The template for the reaction may be cDNA obtained by reverse transcription of mRNA prepared from cells or tissue known to express MEG-7 or MEG-8.

The invention also encompasses (a) DNA vectors that contain any of the foregoing MEG-7 or MEG-8 sequences and/or their complements (i.e., antisense); (b) DNA expression vectors that contain any of the foregoing MEG-7 or MEG-8 sequences operatively associated with a regulatory element that directs the expression of the MEG-7 or MEG-8 coding sequences; and (c) genetically engineered host cells that contain any of the foregoing MEG-7 or MEG-8 sequences operatively associated with a regulatory element that directs the expression of the MEG-7 or MEG-8 coding sequences in the host cell. As used herein, regulatory elements include but are not limited to inducible and non-inducible promoters, enhancers, operators and other elements known to those skilled in the art that drive and regulate expression. Further, the MEG-7 or MEG-8 nucleic acids may be linked to other molecules of interest including, but not limited to, detectable labels, such as radioisotope-containing nucleotides or biotin.

### 5.3. MEG-7 AND MEG-8 Proteins and Polypeptides

MEG-7 and MEG-8 peptides and peptide fragments, mutated, truncated or deleted forms, or fusion proteins comprising MEG-7 and/or MEG-8 peptides can be prepared for a variety of uses, including but not limited to the generation of antibodies, the identification of other cellular gene products involved in MEG-7 or MEG-8 mediated metastatic activity, and the screening for compounds that can be used to modulate such activity.

The present invention provides for the following MEG-7 and MEG-8 peptides:

MEG-7 READING FRAME-I
MQGQAPSSPQFPSAVGSGLSCLHHLRMSLH*LQDRCWDAWAWPHVTCTEL
CLQHKLSYINTVTGI*CX
MEG-7 READING FRAME-II
CRVRPPPLPSFPLLWVLGCHVSTT*GCLYTDFRIDAGMPGHGHMLHVQNF
VYSTN*VI*TQ*LVFNAX
MEG-7 READING FRAME-III
AGSGPLLSPVSLCCGFWAVMSPPLKDVFTLTSG*MLGCLGMATCYMYRTL
STAQIKLYKHSDWYLMX
MEG-8 READING FRAME-I
RLVVLSDSRV*SK*R*LLTKKKKKX
MEG-8 READING FRAME-II
D*WYCLTHVCDPNKGSC*PKKKKKX
MEG-8 READING FRAME-III
ISGTV*LTCVIQIKVAADQKKKKX
*= STOP CODON

While the MEG-7 and MEG-8 peptides can be chemically synthesized (e.g., see Creighton, 1983, Proteins: Structures and Molecular Principles, W.H. Freeman & Co., N.Y.), large polypeptides derived from MEG-7 or MEG-8 and the full length MEG-7 and MEG-8 itself may be advantageously produced by recombinant DNA technology using techniques well known in the art for expressing a nucleic acid containing MEG-7 or MEG-8 gene sequences and/or coding sequences. Such methods can be used to construct expression vectors containing the MEG-7 and MEG-8 nucleotide sequences described in Section 5.1 and appropriate transcriptional and translational control signals. These methods include, for example, in vitro recombinant DNA techniques, synthetic techniques, and in vivo genetic recombination. (See, for example, the techniques described in Sambrook et al., 1989, supra, and Ausubel et al., 1989, supra).

A variety of host-expression vector systems may be utilized to express the MEG-7 and MEG-8 nucleotide sequences of the invention. Where the MEG-7 or MEG-8 peptide or polypeptide is expressed as a soluble derivative and is not secreted, the peptide or polypeptide can be recovered from the host cell. Alternatively, where the MEG-7 or MEG-8 peptide or polypeptide is secreted the peptide or polypeptides may be recovered from the culture media. However, the expression systems also include engineered host cells that express MEG-7 or MEG-8 or functional equivalents. Purification or enrichment of the MEG-7 or MEG-8 from such expression systems can be accomplished using appropriate detergents and lipid micelles and methods well known to those skilled in the art. Such engineered host cells themselves may be used in situations where it is important not only to retain the structural and functional characteristics of the MEG-7 and MEG-8 genes, but to assess biological activity, i.e., in drug screening assays.

The expression systems that may be used for purposes of the invention include but are not limited to microorganisms such as bacteria transformed with recombinant bacteriophage, plasmid or cosmid DNA expression vectors containing MEG-7 or MEG-8 nucleotide sequences; yeast transformed with recombinant yeast expression vectors containing MEG-7 or MEG-8 nucleotide sequences or mammalian cell systems harboring recombinant expression constructs containing promoters derived from the genome of mammalian cells or from mammalian viruses.

Appropriate expression systems can be chosen to ensure that the correct modification, processing and sub-cellular localization of the MEG-7 and MEG-8 proteins occur. To this end, eukaryotic host cells which possess the ability to properly modify and process the proteins are preferred. For long-term, high yield production of recombinant MEG proteins, such as that desired for development of cell lines for screening purposes, stable expression is preferred. Rather than using expression vectors which contain origins of replication, host cells can be transformed with DNA controlled by appropriate expression control elements and a selectable marker gene, i.e., tk, hgprt, dhfr, neo, and hygro gene, to name a few. Following the introduction of the foreign DNA, engineered cells may be allowed to grow for 1-2 days in enriched media, and then switched to a selective media. Such engineered cell lines may be particularly useful in screening and evaluation of compounds that modulate the endogenous activity of the MEG-7 or MEG-8 gene products.

MEG-7 and MEG-8 proteins or peptides may be used to generate antibodies directed against such proteins or peptides

### 5.4. Antibodies to MEG-7 and MEG-8 Peptides

Antibodies that specifically recognize one or more epitopes of MEG-7 or MEG-8, or epitopes of conserved variants of MEG-7 or MEG-8, or peptide fragments of MEG-7 or MEG-8 are also encompassed by the invention. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in conjunction with compound screening schemes, as described, below, in Section 5.5, for the evaluation of the effect of test compounds on expression and/or activity of the MEG-7 or MEG-8 gene products. Also, the antibodies of the invention may be used to diagnose metastatic disease in a subject, or treat such disease.

For production of antibodies, various host animals may be immunized by injection with a MEG-7 or MEG-8 peptides, or a MEG-7 or MEG-8 peptide fused to a carrier protein. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (Bacille Calmette-Guerin) and Corynebacterium parvum.

Polyclonal antibodies comprising heterogeneous populations of antibody molecules may be derived from the sera of the immunized animals. Monoclonal antibodies may be obtained by any technique which provides for the production of antibody molecules by continuous cell lines in culture. These include, but are not limited to, the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclasses thereof. The hybridoma producing the mAb of this invention may be cultivated in vitro or in vivo. Production of high titers of Mabs in vivo makes this the presently preferred method of production.

In addition, techniques developed for the production of “chimeric antibodies” by splicing the genes from a mouse antibody molecule of appropriate antigen specificity together with genes from a human antibody molecule of appropriate biological activity can be used (Morrison et al., 1984, Proc. Nat'l. Acad. Sci., 81:6851-6855; Neuberger et al., 1984, Nature, 312:604-608; Takeda et al. 1985, Nature 314:452-454). Alternatively, techniques developed for the production of humanized antibodies (U.S. Pat. No. 5,585,089) or single chain antibodies (U.S. Pat. No. 4,946,778 Bird, 1988, Science 242:423-426; Huston et al., 1988, Proc. Natl. Acad. Sci USA, 85:5879-5883; and Ward et al., 1989, Nature 334:544-546) may be used to produce antibodies that specifically recognize one or more epitopes of MEG-7 or MEG-8.

### 5.5. Diagnostic Methods

The present invention provides methods and compositions for measuring the invasive and metastatic potential of cancer cells, and for determining whether metastatic cancer cells are present in the subject, based on increased expression of the MEG genes, wherein increased expression of one or more MEG genes supports, indicates, and is consistent with metastatic disease, cancer invasiveness, and/or the likelihood of a cancer to metastasize. Polynucleotide probes, polypeptides and antibodies prepared from the MEG genes (MEG-1 through MEG-8) may be used in assays designed to determine the invasive and/or metastatic potential of primary and secondary neoplasms. Such assays include, but are not limited to Northern blot analysis, PCR, in situ hybridization and immunohistochemistry. Such assays may also include diagnostic imaging of invasive or metastatic cancers. The invention also provides kits comprising one or more ingredients for detecting the levels of MEG gene/protein expression.

The invention provides a method of detecting MEG gene expression in a biological sample, such as a tissue sample. The tissue sample can be, for example, a solid tissue or a fluid sample. A tissue sample in which expression of one or more of the MEG genes/proteins is increased as compared to a control tissue sample, is identified as metastatic or as having metastatic potential. For use as controls, tissue samples can be isolated from other humans, other non-cancerous organs of the patient being tested. Overexpression of one or more of the MEG-1 through MEG-8 proteins or nucleic acids can be detected in the tissue sample.

In one embodiment, the tissue sample is assayed for the over expression of one or more of the MEG proteins. MEG proteins can be detected using MEG protein-specific antibodies. The antibodies can be labeled, for example, with a radioactive, fluorescent, biotinylated, or enzymatic tag and detected directly, or can be detected using indirect immunochemical methods, using a labeled secondary antibody. The expression of the MEG proteins can be assayed, for example, in tissue sections by immunocytochemistry, or in lysates, using Western blotting, as is known in the art.

In another embodiment, the tissue sample is assayed for the expression of MEG mRNAs. MEG mRNAs can be detected by in situ hybridization in tissue sections or in Northern blots containing poly A+ mRNA. MEG-specific probes may be generated using the cDNA sequences known in the art for the MEG-1 through MEG-6 genes or those disclosed herein for the MEG-7 and MEG-8 genes. The probes are preferably 15 to 50 nucleotides in length. The probes can be synthesized chemically or can be generated from longer polynucleotides using restriction enzymes. The probes can be labeled, for example, with a radioactive, biotinylated, or fluorescent tag. If desired, the tissue sample can be subjected to a nucleic acid amplification process.

### 5.6. Inhibitors of MEG Activity

The present invention also contemplates treating metastatic cancers and tumors by inactivating, destroying or nullifying a MEG gene or protein, or cells expressing the MEG gene.

In an embodiment of the invention, antibodies may be utilized to inactivate MEG protein expressing cells: either unconjugated anti-MEG antibodies or anti-MEG antibodies conjugated to a toxin may be employed in the therapy of cancer.

The present invention also provides methods for inhibiting the metastatic activity of cancer cells that is based on inhibition of MEG gene expression. The present invention is based on the discovery that increased MEG gene expression correlates with increased metastatic activity of cancer cells. Such inhibition may be effected through the use of compounds that inhibit the activity of the MEG proteins or MEG gene expression, MEG ribozymes, antisense and siRNA molecules.

In one embodiment of the invention, expression of MEG genes whose expression is upregulated in metastatic cancer can be decreased using a ribozyme, i.e., an RNA molecule with catalytic activity. See, e.g., Cech, 1987, Science 236:1532-1539; Cech, 1990, Ann. Rev. Biochem. 59:543-568; Cech, 1992, Curr. Opin. Struct. Biol. 2:605-609; Couture and Stinchcomb, 1996, Trends Genet. 12:510-515. Ribozymes can be used to inhibit MEG gene function by cleaving an RNA sequence, as is known in the art (e.g., Haseloff et al., U.S. Pat. No. 5,641,673).

The coding sequence of the MEG genes can be used to generate a ribozyme which will specifically bind to mRNA transcribed from the MEG genes. Methods of designing and constructing ribozymes which can cleave other RNA molecules in trans in a highly sequence specific manner have been developed and described in the art (see Haseloff et al. (1988), Nature 334:585-591).

Expression of the MEG genes can also be altered using an antisense oligonucleotide sequence. The antisense sequence is complementary to at least a portion of the coding sequence of a MEG gene. Preferably, the antisense oligonucleotide sequence is at least six nucleotides in length, but can be up to about 50 nucleotides long. Longer sequences can also be used.

The antisense oligonucleotides of the invention can be comprised of DNA, RNA, or any modifications or combinations thereof. As an example of the modifications that the oligonucleotides may contain, inter-nucleotide linkages other than phosphodiester bonds, such as phosphorothioate, methylphosphonate, methylphosphodiester, phosphorodithioate, phosphoramidate, phosphotriester, or phosphate ester linkages (Uhlman et al., Chem. Rev. 90(4):544-584, 1990; Anticancer Research 10:1169, 1990), may be present in the oligonucleotides, resulting in their increased stability. Oligonucleotide stability may also be increased by incorporating 3′-deoxythymidine or 2′-substituted nucleotides (substituted with, e.g., alkyl groups) into the oligonucleotides during synthesis, by providing the oligonucleotides as phenylisourea derivatives, or by having other molecules, such as aminoacridine or poly-lysine, linked to the 3′ ends of the oligonucleotides (see, e.g., Anticancer Research 10:1169-1182, 1990). Modifications of the RNA and/or DNA nucleotides comprising the oligonucleotides of the invention may be present throughout the oligonucleotide, or in selected regions of the oligonucleotide, e.g., the 5′ and/or 3′ ends. The antisense oligonucleotides may also be modified so as to increase their ability to penetrate the target tissue by, e.g., coupling the oligonucleotides to lipophilic compounds. The antisense oligonucleotides of the invention can be made by any method known in the art, including standard chemical synthesis, ligation of constituent oligonucleotides, and transcription of DNA encoding the oligonucleotides, as described below.

Precise complementarity is not required for successful duplex formation between an antisense molecule and the complementary coding sequence of a MEG gene. Antisense molecules which comprise, for example, 2, 3, 4, or 5 or more stretches of contiguous nucleotides which are precisely complementary to a portion of a coding sequence of a MEG gene, each separated by a stretch of contiguous nucleotides which are not complementary to adjacent coding sequences, can provide targeting specificity for mRNA of a MEG gene. Preferably, each stretch of contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular MEG gene coding sequence.

In yet another embodiment of the invention dsRNA-mediated interference (RNAi) which is well known in the fields of molecular biology, may be used to inhibit the expression of the MEG genes (see, for example, C. P. Hunter, Current Biology, 1999, 9:R440-442; Hamilton et al., 1999, Science 286:950-952; and S. W. Ding, Current Opinions in Biotechnology, 2000, 11:152-156, hereby incorporated by reference in their entireties). RNAi typically comprises a polynucleotide sequence identical or homologous to a target gene (or fragment thereof) linked directly, or indirectly, to a polynucleotide sequence complementary to the sequence of the target gene (or fragment thereof). The dsRNA may comprise a polynucleotide linker sequence of sufficient length to allow for the two polynucleotide sequences to fold over and hybridize to each other; however, a linker sequence is not necessary. The linker sequence is designed to separate the antisense and sense strands of RNAi significantly enough to limit the effects of steric hindrances and allow for the formation of dsRNA molecules and should not hybridize with sequences within the hybridizing portions of the dsRNA molecule. Accordingly, one method for inhibiting the metastatic activity of cancer cells comprises the use of (RNAi) comprising polynucleotide sequences identical or homologous to one or more of the MEG genes.

RNA containing a nucleotide sequence identical to a fragment of the target gene is preferred for inhibition; however, RNA sequences with insertions, deletions, and point mutations relative to the target sequence can also be used for inhibition. As described above for anti sense molecules, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

Preferably and most conveniently, RNAi is targeted to a polynucleotide sequence, such as the MEG-1 through MEG-8 genes. Preferred RNAi molecules of the instant invention are highly homologous or identical to the polynucleotides encoding the MEG-1 through MEG-8 genes. The homology may be greater than 70%, preferably greater than 80%, more preferably greater than 90% and is most preferably greater than 95%.

Ribozymes, antisense polynucleotides, and RNAi molecules may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands); the promoters may be known inducible promoters such as baculovirus. Inhibition may be targeted by specific transcription in an organ, tissue, or cell type. The RNA strands may or may not be polyadenylated; the RNA strands may or may not be capable of being translated into a polypeptide by a cell's translational apparatus.

RNA may also be chemically or enzymatically synthesized by manual or automated reactions. The RNA may be synthesized by a cellular RNA polymerase or a bacteriophage RNA polymerase (e.g., T3, T7, SP6). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no, or a minimum of, purification to avoid losses due to sample processing. The RNA may be dried for storage or dissolved in an aqueous solution. The solution may contain buffers or salts to promote annealing, and/or stabilization of the duplex strands.

Ribozymes, antisense molecules, and RNAi can be introduced into cells as part of a DNA construct, as is known in the art. The DNA construct can also include transcriptional regulatory elements, such as a promoter element, an enhancer or UAS element, and a transcriptional terminator signal, for controlling the transcription of the ribozyme in the cells. Mechanical methods, such as microinjection, liposome-mediated transfection, electroporation, or calcium phosphate precipitation, can be used to introduce such DNA constructs into cells whose division it is desired to decrease, as described above. Alternatively, if it is desired that the DNA construct be stably retained by the cells, the DNA construct can be supplied on a plasmid and maintained as a separate element or integrated into the genome of the cells, as is known in the art.

### 5.7. Compositions Containing Modulators of MEG Activity and Their Uses

The present invention provides for methods of inhibiting the metastatic activity of cancer cells of a subject, comprising administering to the subject an effective amount of a MEG inhibitor. An “effective amount” of the MEG inhibitor is an amount that decreases the metastatic activity of cancer cells.

The active ingredients of a pharmaceutical composition containing the anti-MEG antibodies and antisense MEG nucleic acids (i.e., anti-cancer reagents) are contemplated to exhibit effective therapeutic activity, for example, for treating cancer. Thus, the active ingredients of the therapeutic compositions containing anti-cancer reagents are administered in therapeutic amounts which depend on the particular disease. The dosage regimen may be adjusted to provide the optimum therapeutic response. For example, several divided doses may be administered daily or the dose may be proportionally reduced as indicated by the exigencies of the therapeutic situation. A decided practical advantage is that the active compound may be administered in a convenient manner such as by the oral, intravenous (where water soluble), intramuscular, subcutaneous, intranasal, intradermal or suppository routes. Depending on the route of administration, the active ingredients which comprise MEG anti-cancer reagents may be required to be coated in a material to protect said ingredients from the action of enzymes, acids and other natural conditions which may inactivate said ingredients.

The active compounds may also be administered parenterally or intraperitoneally. Dispersions can also be prepared in glycerol, liquid polyethylene glycols, and mixtures thereof, and in oils. Under ordinary conditions of storage and use, these preparations contain a preservative to prevent the growth of microorganisms.

As used herein, “pharmaceutically acceptable carrier” includes any and all solvents, dispersion media, coatings, antibacterial and antifungal agents, isotonic and adsorption delaying agents, and the like. The use of such media agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

### 5.8. Diagnostic Methods Using MDA-9/Syntenin

The present invention provides methods and compositions for measuring the invasive and metastatic potential of cancer cells based on increased expression of the mda-9/syntenin gene, where mda-9 is not a MEG gene, as defined herein. The complete amino acid sequence of MDA-9 and of the MDA-9 gene is set forth in U.S. Pat. No. 6,548,650. Polynucleotide probes, polypeptides and antibodies prepared from the mda-9/syntenin gene may be used in assays designed to determine the invasive and/or metastatic potential of primary and secondary neoplasms. Such assays include, but are not limited to Northern blot analysis, PCR, in situ hybridization and immunohistochemistry. Such assays may also include diagnostic imaging of invasive or metastatic cancers. The invention also provides kits comprising one or more ingredients for detecting the level of the mda-9/syntenin gene or protein expression.

The invention provides a method of detecting mda-9/syntenin gene expression in a biological sample, such as a tissue sample. The tissue sample can be, for example, a solid tissue or a fluid sample. A tissue sample in which expression the mda-9/syntenin gene or protein is increased as compared to a control tissue sample, is identified as metastatic or as having metastatic potential. For use as controls, tissue samples can be isolated from other humans, other non-cancerous organs of the patient being tested. Overexpression of the mda-9/syntenin protein or nucleic acid can be detected in the tissue sample.

In one embodiment, the tissue sample is assayed for the over expression of the mda-9/syntenin protein. mda-9/syntenin protein can be detected using mda-9/syntenin protein-specific antibodies. The antibodies can be labeled, for example, with a radioactive, fluorescent, biotinylated, or enzymatic tag and detected directly, or can be detected using indirect immunochemical methods, using a labeled secondary antibody. The expression of the mda-9/syntenin protein can be assayed, for example, in tissue sections by immunocytochemistry, or in lysates, using Western blotting, as is known in the art.

In another embodiment, the tissue sample is assayed for the expression of mda-9/syntenin mRNA. mda-9/syntenin mRNA can be detected by in situ hybridization in tissue sections or in Northern blots containing poly A+ mRNA. mda-9/syntenin-specific probes may be generated using the cDNA sequences known in the art for the mda-9/syntenin gene. The probes are preferably 15 to 50 nucleotides in length. The probes can be synthesized chemically or can be generated from longer polynucleotides using restriction enzymes. The probes can be labeled, for example, with a radioactive, biotinylated, or fluorescent tag. If desired, the tissue sample can be subjected to a nucleic acid amplification process.

### 5.9. Inhibitors of MDA-9/Syntenin Activity

The present invention also contemplates treating metastatic cancers and tumors by inactivating, destroying or nullifying the mda-9/syntenin gene or protein, or cells expressing the mda-9/syntenin gene or protein (mda-9/syntenin is not a MEG gene, as defined herein).

In an embodiment of the invention, antibodies may be utilized to inactivate the mda-9/syntenin protein expressing cells: either unconjugated anti-the mda-9/syntenin antibodies or anti-the mda-9/syntenin antibodies conjugated to a toxin may be employed in the therapy of cancer.

The present invention also provides methods for inhibiting the metastatic activity of cancer cells that is based on inhibition of mda-9/syntenin gene expression. The present invention is based on the discovery that increased mda-9/syntenin gene expression correlates with increased metastatic activity of cancer cells. Such inhibition may be effected through the use of compounds that inhibit the activity of the mda-9/syntenin protein or mda-9/syntenin gene expression, mda-9/syntenin ribozymes, antisense and siRNA molecules.

In one embodiment of the invention, expression of the mda-9/syntenin gene whose expression is upregulated in metastatic cancer can be decreased using a ribozyme, i.e., an RNA molecule with catalytic activity. See, e.g., Cech, 1987, Science 236:1532-1539; Cech, 1990, Ann. Rev. Biochem. 59:543-568; Cech, 1992, Curr. Opin. Struct. Biol. 2:605-609; Couture and Stinchcomb, 1996, Trends Genet. 12:510-515. Ribozymes can be used to inhibit the mda-9/syntenin gene function by cleaving an RNA sequence, as is known in the art (e.g., Haseloff et al., U.S. Pat. No. 5,641,673).

The coding sequence of the mda-9/syntenin gene can be used to generate a ribozyme which will specifically bind to mRNA transcribed from the mda-9/syntenin gene. Methods of designing and constructing ribozymes which can cleave other RNA molecules in trans in a highly sequence specific manner have been developed and described in the art (see Haseloff et al. (1988), Nature 334:585-591).

In yet another embodiment of the invention dsRNA-mediated interference (RNAi or siRNA) which is well known in the fields of molecular biology, may be used to inhibit the expression of the mda-9/syntenin gene (see, for example, C. P. Hunter, Current Biology, 1999, 9:R440-442; Hamilton et al., 1999, Science 286:950-952; and S. W. Ding, Current Opinions in Biotechnology, 2000, 11:152-156, hereby incorporated by reference in their entireties). RNAi typically comprises a polynucleotide sequence identical or homologous to a target gene (or fragment thereof) linked directly, or indirectly, to a polynucleotide sequence complementary to the sequence of the target gene (or fragment thereof). The dsRNA may comprise a polynucleotide linker sequence of sufficient length to allow for the two polynucleotide sequences to fold over and hybridize to each other; however, a linker sequence is not necessary. The linker sequence is designed to separate the antisense and sense strands of RNAi significantly enough to limit the effects of steric hindrances and allow for the formation of dsRNA molecules and should not hybridize with sequences within the hybridizing portions of the dsRNA molecule. Accordingly, one method for inhibiting the metastatic activity of cancer cells comprises the use of siRNA comprising polynucleotide sequences identical or homologous to the mda-9/syntenin gene. Specific examples of SiRNA molecules found to be effective in inhibiting migration of cancer cells are described in Section 7, below. The present invention provides for additional molecules that are at least 90 to 95 percent homologous to SEQ ID NO:3 or SEQ ID NO:4 using standard software such as BLAST or FASTA.

RNA containing a nucleotide sequence identical to a fragment of the target gene is preferred for inhibition; however, RNA sequences with insertions, deletions, and point mutations relative to the target sequence can also be used for inhibition. Sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

Preferably and most conveniently, RNAi is targeted to a polynucleotide sequence, of the mda-9/syntenin gene. Preferred RNAi molecules of the instant invention are highly homologous or identical to the polynucleotides encoding the mda-9/syntenin gene. The homology may be greater than 70%, preferably greater than 80%, more preferably greater than 90% and is most preferably greater than 95%.

Ribozymes, antisense polynucleotides, and RNAi molecules may be synthesized either in vivo or in vitro or introduced into cells as described in paragraphs [0074-0076].

### 5.10. Compositions Containing Modulators of MDA-9/Syntenin Activity and Their Uses

The present invention provides for methods of inhibiting the metastatic activity of cancer cells of a subject, comprising administering to the subject an effective amount of a mda-9/syntenin inhibitor. An “effective amount” of the mda-9/syntenin inhibitor is an amount that decreases the metastatic activity of cancer cells.

The active ingredients of a pharmaceutical composition containing the anti-mda-9/syntenin antibodies or siRNA mda-9/syntenin nucleic acids (i.e., anti-cancer reagents) are contemplated to exhibit effective therapeutic activity, for example, for treating cancer. Thus, the active ingredients of the therapeutic compositions containing anti-cancer reagents are administered in therapeutic amounts which depend on the particular disease. The dosage regimen may be adjusted to provide the optimum therapeutic response. For example, several divided doses may be administered daily or the dose may be proportionally reduced as indicated by the exigencies of the therapeutic situation. A decided practical advantage is that the active compound may be administered in a convenient manner such as by the oral, intravenous (where water soluble), intramuscular, subcutaneous, intranasal, intradermal or suppository routes. Depending on the route of administration, the active ingredients which comprise the mda-9/syntenin anti-cancer reagents may be required to be coated in a material to protect said ingredients from the action of enzymes, acids and other natural conditions which may inactivate said ingredients.

The active compounds may also be administered parenterally or intraperitoneally. Dispersions can also be prepared in glycerol, liquid polyethylene glycols, and mixtures thereof, and in oils. Under ordinary conditions of storage and use, these preparations contain a preservative to prevent the growth of microorganisms.

As used herein, “pharmaceutically acceptable carrier” includes any and all solvents, dispersion media, coatings, antibacterial and antifungal agents, isotonic and adsorption delaying agents, and the like. The use of such media agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

### 6. EXAMPLE

### 6.1. Materials and Methods

### 6.1.1. Human Melanoma Cells

The M4Beu melanoma cell line was established from a lymph node metastasis of a patient with malignant melanoma (Boukerche et al., 2000, Cancer Res. 60:5848-5856). Highly metastatic 7GP122 and T1P26R melanoma cell lines derived from these parental cells are variants obtained by successive orthotopic transplantations of M4Beu tumors or lymph node metastases in immunosuppressed newborn rats (Boukerche et al., 2000, supra; Baril et al., 2002, Int. J. Cancer 99:315-22). FM516-SV (FM516) is a normal human melanocyte culture immortalized by the SV40 T-antigen gene (Melber et al., 1989, Cancer Res. 49:3650-3655). Additional melanoma cell lines established from patients with metastatic melanomas that were evaluated include WM239, C8161, and MeWo (Jiang et al., 2000, Proc. Natl. Acad. Sci. U.S.A. 97:12684-12689). The cells were routinely cultured as monolayers in RPMI-1640 or Dulbecco's modified Eagle's medium supplemented with 10% fetal calf serum, 100 IU/ml penicillin and 100 IU/ml streptomycin. They are regularly screened for mycoplasma contamination using the BM-Cyclin procedure (Roche Diagnostics).

### 6.1.2. Metastasis Assay

The tumorigenicity and spontaneous metastatic ability during the course of the experiments were evaluated in immunosuppressed newborn rats as previously described (Boukerche et al., 1994, Eur. J. Biochem. 220:485-491; Baril et al., 2002, Int. J. Cancer 99:315-22). Briefly, Wistar rats less than 24 hours old received on day 0 a subcutaneous (s.c.) injection of 10⁶ cells in 0.1 ml phosphate buffer in the abdomen, followed by a s.c. injection of an optimal dose of anti-thymocyte serum (ATS) in the dorsum on days 0, 2, 7 and 14. Animals were sacrificed on day 21 and the metastatic potential was determined by direct counting of the pulmonary nodules.

### 6.1.3. RNA Isolation for cDNA Subtraction and Northern Blotting

Total RNA was extracted from melanoma cells by using the RNAsy Maxi kit (Qiagen). mRNA was prepared from total RNA with the Gibco BRL MessageMaker mRNA Isolation System as described in the product profile.

### 6.1.4. RaSH Procedure

The sequences of oligonucleotides that were used are as follows: XDNP-18 CTGATCACTCGAGAGATC, (SEQ ID NO: 5) XDPN-14 CTGATCACTCGAGA, (SEQ ID NO: 6) XDPN-12 GATCTCTCGAGT (SEQ ID NO: 7). The adapters formed from the two sets of oligonucleotides contained an XhoI recognition site.

To clone cDNAs expressed at elevated levels in T1P26R melanoma cell line, 1 μg of poly(A) RNA from the poorly metastatic M4Beu cell line (driver) or highly metastatic variant T1P26R (tester) was used for double-stranded cDNAs synthesis (Gubler and Hoffman, 1983). The cDNAs were subsequently digested with DpnII at 37° C. for 3 h. followed by phenol/chloroform extraction and ethanol precipitation. The resulting cDNA fragments were each ligated with primers XDPN-12/XDPN14 (final concentration 20 μM) in 30 μl of 1X ligation buffer (Gibco), heated at 55° C. for 1 min, and cooled down to 14° C. within 1 h. After adding 3 μl of T4 DNA ligase (5U/μl) (Gibco) to the mixtures individually, ligation was performed at 14° C. overnight. The mixtures were diluted to 100 μl with TE buffer (pH 7.0), and at least 40 μl of the mixtures were used for PCR amplification as follow: 1 μl of the cDNA mixture, 10 μl 10×PCR buffer, 1 mM MgCl₂, 0.4 mM dNTPs, 1 μM XDPN18, and 1 U Taq polymerase (Gibco). Amplification was performed with the following cycling parameters: 1 min at 94° C., 25 cycles at 94° C. for 30 s; 55° C. for 1 min, 72° C. for 1 min followed by one cycle for 3 min at 72° C., and a final extension at 72° C. for 5 min. The PCR products were pooled and purified using RNAsy Maxi Kit (Qiagen). Ten μg of the tester PCR products were then digested with XhoI followed by phenol/chloroform extraction and ethanol precipitation.

One hundred ng of the digested tester cDNA were mixed with an excess of driver cDNA (3 μg), boiled for 5 min and hybridized in 20 μl hybridization buffer (50 mM HEPES, pH 7.5, 500 mM NaCl, 0.2% SDS, 40% formamide) for 48 h at 42° C. The hybridization mixture was subjected to a phenol/chloroform extraction, precipitated in ethanol and then dissolved in 20 μl of TE buffer. One μl of the subtracted cDNAs was ligated onto the XhoI digested, CIP-treated pBluescript II SK plasmid (Stratagene, La Jolla, Calif., USA), overnight at 14° C., and transformed into competent cells, Escherichia coli DH5α.

Bacterial plasmids from individual colonies were randomly isolated and PCR amplified. The PCR products were blotted onto filters and reverse northern blotting using radioactive probes generated from poly (A) RNA isolated from M4Beu or T1P26R cells were performed to identify cDNAs displaying differential expression in both cell lines (Kang et al., 1998 Proc. Natl. Acad. Sci. U.S.A. 95:13788-13793; Huang et al., 1999 Gene 236:125-131; Jiang et al., 2000, Proc. Natl. Acad. Sci. U.S.A. 97:12684-12689). cDNAs displaying elevated expression in T1P26R cells were designated metastasis-elevated genes (MEGs) clone 1 to 8.

Appropriate expression of the MEGs identified by reverse Northern blotting was confirmed by Northern blotting as follows: 10 μg of total RNA samples were resolved by formaldehyde gel electrophoresis, transferred to positively charged nylon membranes, and then cross-linked by UV light. Specific probes were generated by labeling differentially expressed fragments with α³²P-dCTP using a random labeling kit (Boehringer Mannheim). The membranes were prehybridized in ExpressHyb solution (Clontech) for 30 min. at 68° C., then hybridized in the same solution for 2 h at 68° C. The filters were sequentially washed in 2×SSC and 0.01% SDS, and 0.1% SSC and 0.1% SDS at 68° C. The blots were then exposed at −80° C. with two intensifying screens overnight. Filters were stripped and subsequently probed with a glyceraldehyde-3-phosphate dehydrogenase (GAPDH) probe to ensure the presence of equivalent amounts of RNA in each lane of a filter.

Cloned cDNAs that were confirmed to be differentially expressed in the first and the second differential screening step were sequenced with T7 and SP6 primers using automated cycle sequencing at the DNA facility of Columbia University. The obtained partial cDNA sequences were corrected for any pBluescript vector and primer sequences, prior to computer BLASTN search in databases at the National Institute for Biotechnology Information.

### 6.2. Results and Discussion

### 6.2.1. Experimental Model System to Define Gene Changes Associated with the Early Changes in Melanoma Metastasis Progression

Instead of starting directly from human tumor material, a defined series of variants selected from a poorly metastatic human melanoma cell line M4Beu that generate a higher frequency of spontaneous lung metastases in immunosuppressed neonatal rats were chosen (Boukerche et al., 1994, Eur. J Biochem. 220:485-491; Baril et al., 2002, Int. J. Cancer 99:315-22). A potential advantage of this strategy is that it overcomes some of the problems existing when dealing with primary tumor samples, such as tumor heterogeneity, individual tissue variations, and difficulty in obtaining pure tumor cells from tissue samples with normally complex histological organization. In addition, the assumption that the gene expression pattern of tumor cell lines does not significantly differ from the tissue it was originally derived from is supported by the observations of Ross et al. 2000, Nat. Genet. 24:227-235 showing that in the 60 cell lines studied by cDNA array analyses, neither physiological nor experimental adaptation for growth in culture was sufficient to significantly alter the gene expression programs established during differentiation in vivo.

The human parental melanoma cell line M4Beu and its selected metastatic variant T1P26R were used to isolate potential melanoma progression genes. Before subtraction, the phenotype of the cell lines were verified by subcutaneous injections of each cell line into antithymocyte immunosuppressed newborn rats. All inoculated animals developed subcutaneous tumors after injection of 2×10⁶ cells per animal. However, while the M4Beu parental melanoma cell line expresses very low, if not at all, metastatic potential, the melanoma variants T1P26R and 7GP122 have higher metastatic potential, mostly restricted to lungs. The parental cell line and the variants have retained their metastatic properties following continuous subculture, suggesting that the changes that had been selected for in vivo represent stable traits not lost upon repeated maintenance in monolayer on plastic substrates.

### 6.2.2. Cloning and Expression Analysis of Metastasis-Elevated Genes (MEGS) Identified Using the RaSH Approach

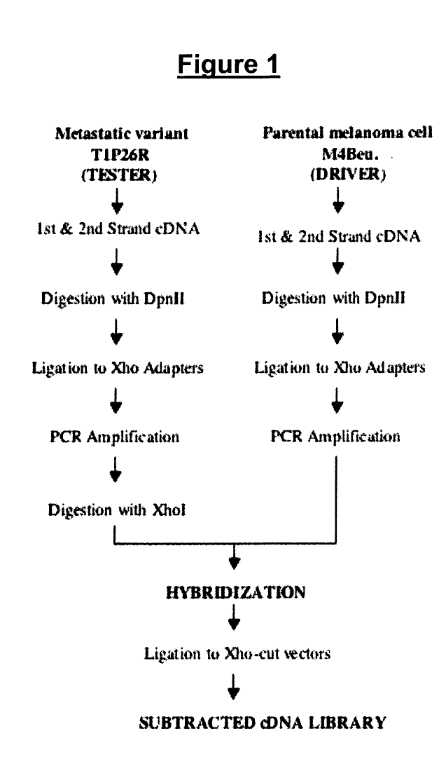

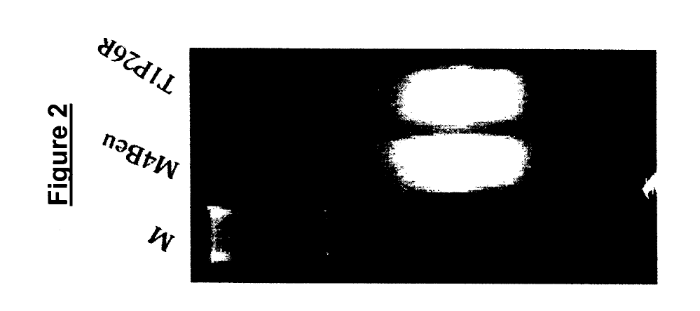

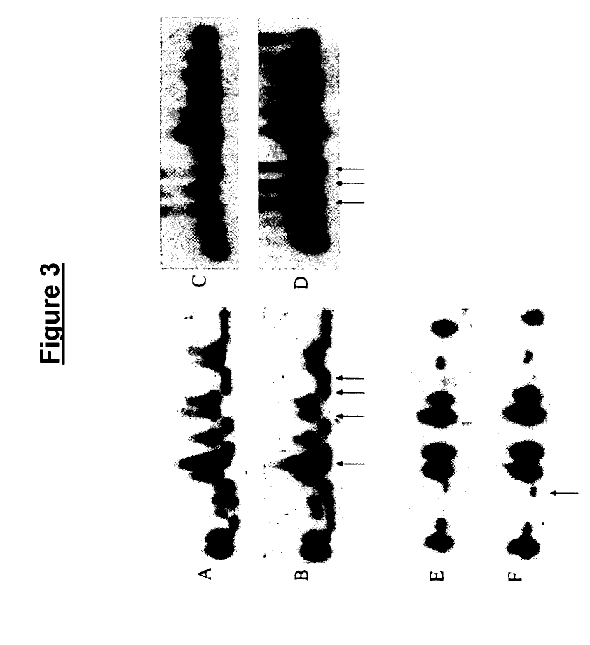

The RaSH approach was performed between the nonmetastatic cell lines M4Beu and the metastatic cell line variant T1P26R to identify differentially expressed genes. A schematic of this approach as applied to this melanoma tumor progression model is shown in FIG. 1. For RaSH, the cDNAs from highly and poorly metastatic cell lines were digested with DpnII, a four-base cutting restriction enzyme that generates cohesive-end cDNA fragments suitable for hybridization. The cDNA fragments from the tester and the driver were then ligated to adaptors and selectively amplified by PCR. As shown in FIG. 2, two sets of cDNA libraries with an average size of 256-bp were generated. Subtraction hybridization was then performed by incubating the tester (highly metastatic cell line library) and driver (poorly metastatic library) PCR fragments without additional PCR amplification steps. Selection of subtracted cDNAs was achieved by matching the ends of the cDNA fragments to the ends of the plasmid vectors during ligation and construction of subtracted libraries. Colonies from the subtracted library were then isolated randomly and the PCR-products ranging from 250-800 bp were initially screened by reverse Northern hybridization. Of the 72 genes analyzed, only 8 MEGs were detected as overexpressed in the metastatic variant with respect to the nonmetastatic cells based on the criteria requiring a minimum of a twofold difference in hybridization signal (FIG. 3). Most of the other clones gave positive signals but did not show overexpression in either cell line, supporting the utility of Reverse Northern as a primary screen for differentially expressed genes identified using the RaSH approach (Jiang et al., 2000). This frequency of differentially expressed genes is significantly less than observed in previous applications, which is ˜50% differentially expressed genes which display concordance between Reverse Northerns and true expression by Northerns (Jiang et al., 2000). This difference in obtaining differentially expressed genes using the RaSH approach most likely reflects the nature of the starting materials, since although TIP26R forms large numbers of metastases, its parental (driver hybridization source) also displays metastatic potential, albeit significantly reduced (Table I).

TABLE I
Metastatic capacity of M4Beu human melanoma cells and nude rat-derived
variants in immunosuppressed newborn rats. One million cells
were injected subcutaneously into newborn rats immunosuppressed
with antithymocytes serum, and tumorigenicity and metastatic
potential were determined after 3 weeks (Boukerche et al., 1994,
Eur. J. Biochem. 220: 485-491).

| | | | |
|---|---|---|---|
| Cell | Primary | Metastasis | Lung nodules/rat |

| lines | Tumors^{a} | incidences^{b} | Range | Median |
|---|---|---|---|---|
| Low Metastatic Melanoma Cell Line | | | | |
| M4Beu | 10/10 | 2/10 | 0-100 | 0 |
| High metastatic Melanoma Variant Cell Lines | | | | |
| T1P26R | 12/12 | 12/12 | 30->>300 | >300 |
| 7GP122 | 10/10 | 10/10 | 10->>200 | 200 |

^{a}Number of newborn rats with primary tumors versus number of newborn rats injected subcutaneously with 1 × 106 melanoma cells.
^{b}Number of newborn rats with lung metastases versus number of newborn rats injected subcutaneously with 1 × 106 melanoma cells.

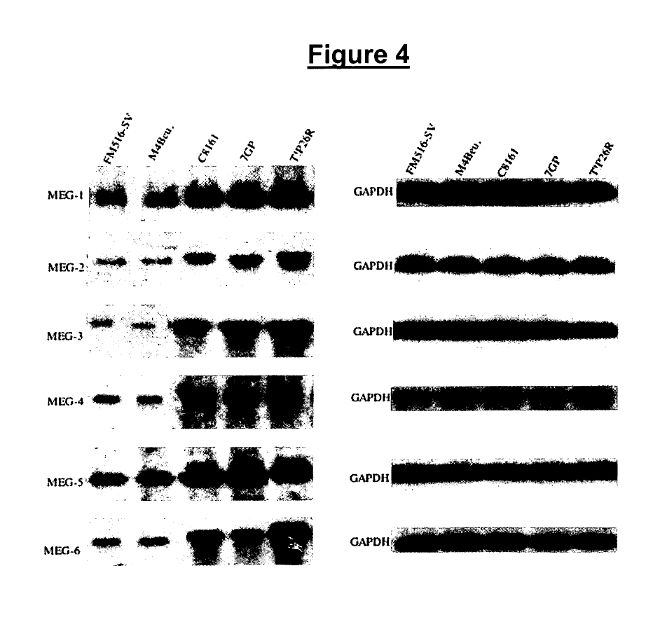

For a gene to be potentially useful as a diagnostic marker, or to be relevant functionally in metastasis, its RNA should be in relative abundance. The eight MEG cDNA clones found to be upregulated by Reverse Northern screen were hybridized to membrane-blotted total RNA isolated from normal SV40-immortalized human melanocytes (FM516-SV) and four melanoma cell lines, M4Beu, T1P26R, 7GP122 and C8161. As shown in FIGS. 4 and 5, most of these MEGs showed true differential expression in the metastatic melanoma variants versus normal immortalized melanocytes and nonmetastatic melanoma cells. Although basal expression levels differed slightly in the nonmetastatic cell line M4Beu, expression of most of the eight MEG genes were up-regulated in all of the metastatic variants, including C8161, a human cutaneous melanoma cell line known to be highly invasive in vitro and in vivo with metastatic potential in animal models (Welch et al., 1991, Int. J. Cancer 2:227-237). These results support a high degree of concordance in expression between Reverse Northern screening and Northern blotting. Moreover, they extend previous observations showing that the absence of PCR amplification in RaSH during subtraction, including the use of different primer designs and subtraction approaches as compared to other PCR-based protocols, significantly reduces false-positive signals (Kang et. al., 2002, Analysing Gene Expression, Ch. 3.2.4.6, pg. 206-214).

Sequencing of the eight differentially expressed MEG clones and comparing these to those deposited in the GenBank and EMBL databases, confirmed that six of the clones (MEG-1 to MEG-6) had significant homology to known DNA sequences (Table 2). In contrast, two sequences (MEG-7 and MEG-8) were designated novel, since no similarity has been found to hypothetical proteins (Table II). The known genes that displayed >95% identity with previously characterized genes, included the 67-kDa laminin receptor (MEG-1), endothelin receptor B (MEG-2), Na⁺/K⁺-ATPase (MEG-3), interleukin-receptor associated kinase known also as IRAK-1 (MEG-4), Ku antigen (MEG-5), and ribosomal protein RPLA37 (MEG-6). Among these known genes, some of them have never been reported to be associated with tumor progression.

TABLE II
MEG cDNA clones isolated using the RaSH protocol.

| | | Approximate Size | Fold-Up | Characteristics |
|---|---|---|---|---|
| Nomenclature^{a} | Identity^{b} | of mRNA (kb) | Regulation^{c} | and Functions |
| MEG-1 | Endothelin | ˜1.7 | 3.5 | Cell-cell |
| | receptor B | | | communication, |
| | | | | Angiogenesis |
| MEG-2 | 67-kDa laminin | ˜1 | 3 | Non-integrin |
| | receptor | | | receptor, |
| | | | | modulates tumor |
| | | | | cell adhesion |
| MEG-3 | Ku antigen | ˜3 | 3.5 | DNA repair |
| | | | | double-strand |
| | | | | breaks (DSBs), |
| | | | | Cell-ECM |
| | | | | interactions |
| MEG-4 | Interleukin- | ˜3.5 | 3.5 | Signal |
| | receptor- | | | transduction |
| | associated kinase | | | through NF-κb |
| | IRAK | | | pathway |
| MEG-5 | Ribosomal protein | ˜0.5 | 4.2 | Metabolic activity |
| | RPLA L37a | | | |
| MEG-6 | Na/K ATPase | ˜2.5 | 4.3 | Cell-ECM |
| | Sodium/potassium | | | interactions |
| | pump | | | |
| MEG-7 | Novel | ˜0.6 | 3.8 | Unknown |
| | | | | function |
| MEG-8 | Novel | ˜0.8 | 2.5 | Unknown |
| | | | | function |

^{a}Clones were designated as Metastasis Elevated Genes (MEG).
^{b}Sequences were searched against various DNA databases to determine identity.
^{c}Relative fold induction for T1P26R melanoma in comparison to FM-516-SV melanocytes. The values were normalized to GAPDH mRNA.

Additionally, two novel cDNA fragments were identified in this study, designated MEG-7 and MEG-8, as candidate molecular markers of early stages of tumor progression. These genes were detected as mRNA transcripts of ˜0.6 and ˜0.8-kb, respectively, in all melanocytic cells tested (FIG. 5), but expression level was greatest (˜2- to 3.8-fold) in metastatic cells compared with normal immortal melanocytes FM516-SV and non-metastatic M4Beu melanoma cells (FIG. 5). Interestingly, T1P26R variant which has been selected in vivo for its high ability to metastasize express higher levels of the MEG-8 mRNA transcript. The finding that expression level of this gene is relatively high in FM516-SV cells suggests that this gene is constitutively expressed in normal melanocytes with its expression level enhanced during tumor progression. Alternatively, moderate mRNA expression of MEG-7 observed in the FM516-SV normal human melanocyte cell line immortalized by the SV40 T-antigen gene may not reflect the situation of normal melanocytes in situ.

### 7. EXAMPLE

### Inhibition of MDA-9/Syntenin Activity Modulates Migratory Activity of Cancer Cells

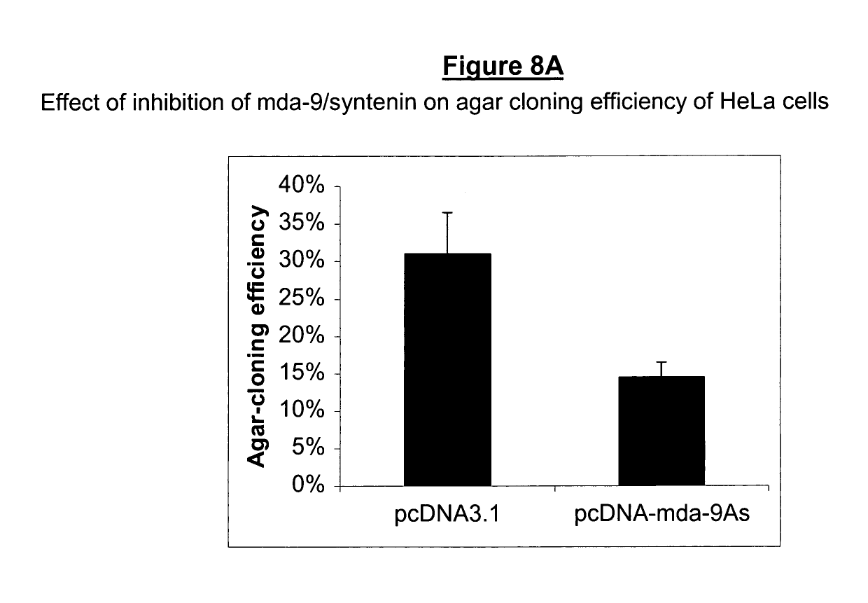

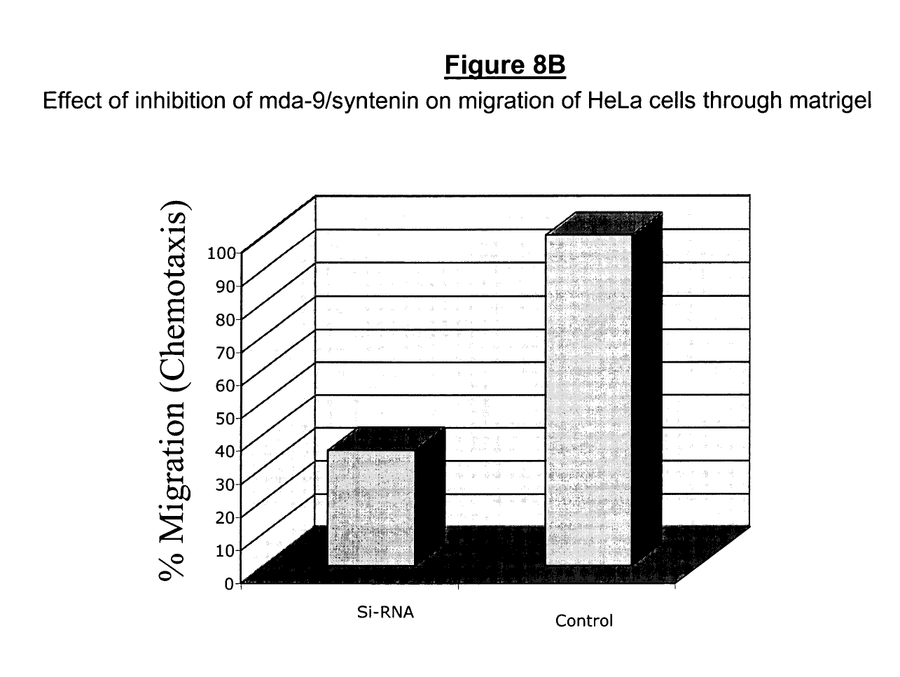

HeLa cells were transfected with empty pcDNA3.1 vector or a plasmid expressing anti-sense mda-9/syntenin (pcDNA-mda-9AS) and anchorage-independent growth in soft agar was analyzed (FIG. 8A). HeLa cells were transfected with either control scrambled siRNA or siRNA against mda-9/syntenin (mda-9 siRNA) and the migration of the cells through Matrigel-coated filters was analyzed (FIG. 8B).

(SEQ ID NO:3)

| | |
|---|---|
| Sense strand siRNA: | CUUGAAGGUAGACAAAGUAtt |

(SEQ ID NO:4)

| | |
|---|---|
| Antisense strand siRNA: | UACUUUGUCUACCUUCAAGtt |

In HeLa cells, which express high levels of mda-9/syntenin, inhibition of mda-9/syntenin, using either an antisense overexpressing plasmid or a siRNA, markedly decreased anchorage-independent growth in soft agar (FIG. 8A) and migration of cells through Matrigel-coated filters (FIG. 8B). This data indicates that modulating expression of mda-9/syntenin, either by direct gene knockdown strategies (antisense or siRNA), or by using small molecule inhibitors provides a means of inhibiting tumor metastasis.

The present invention is not to be limit in scope by the specific embodiments described herein. Indeed, various modifications of the invention in addition to those described herein will become apparent to those skilled in the art from the foregoing description and accompanying figures. Such modifications are intended to fall within the scope of the appended claims.

Various references are cited herein, the disclosures of which are incorporated by reference in their entireties.

We claim

1. An isolated nucleic acid molecule that hybridizes to a nucleic acid molecule having a sequence as set forth in SEQ ID NO:1 under stringent conditions and that encodes a protein having increased expression in metastatic cancer cells as compared to normal cells.

2. An isolated nucleic acid molecule that hybridizes to a nucleic acid molecule having a sequence as set forth in SEQ ID NO:2 under stringent conditions and that encodes a protein having increased expression in metastatic cancer cells as compared to normal cells.

3. A method of inhibiting the metastatic activity of a cell comprising administering to the subject an effective amount of a MEG inhibitor.

4. The method of claim 3 wherein the inhibitor is an antisense molecule.

5. The method of claim 3 wherein the inhibitor is a ribozyme molecule.

6. The method of claim 3 wherein the inhibitor is an interfering RNA (RNAi).

7. The method of claim 3 wherein the inhibitor is an antibody that binds to a MEG protein.

8. A method for determining the metastatic activity of a cancer cell comprising:
(i) determining the level of expression of one or more MEG genes in a sample derived from a subject suspected of having metastatic cancer;
(ii) comparing the level of expression of the one or more MEG genes in the sample to the level of expression in a control sample; and
wherein an increase in the level of expression of a MEG gene in the sample compared to the control indicates that the cancer cell has metastatic activity.

9. The method of claim 8 wherein the MEG gene is MEG-1.

10. The method of claim 8 wherein the MEG gene is MEG-2.

11. The method of claim 8 wherein the MEG gene is MEG-3.

12. The method of claim 8 wherein the MEG gene is MEG-4.

13. The method of claim 8 wherein the MEG gene is MEG-5.

14. The method of claim 8 wherein the MEG gene is MEG-6.

15. The method of claim 8 wherein the MEG gene is MEG-7.

16. The method of claim 8 wherein the MEG gene is MEG-8.

17. A method for inhibiting cancer cell migration, comprising administering, to said cell, an effective amount of an inhibitor of mda-9 expression.

18. The method of claim 17, wherein the inhibitor is siRNA.


SEQUENCE LISTING (7 sequences)

SEQ ID NO: 1 - DNA, 202 bp, Homo sapien
atgcagggtcaggccccctcctctccccagtttccctctgctgtgggttctgggctgtcatgtctccaccacttaaggatgtctttacactgacttcaggatagatgctgggatgcctgggcatggccacatgttacatgtacagaactttgtctacagcacaaattaagttatataaacacagtgactggtatttaatgct

SEQ ID NO: 2 - DNA, 73 bp, Homo sapien
cgattagtggtactgtctgactcacgtgtgtgatccaaataaaggtagctgctgaccaaaaaaaaaaaaaaaa

SEQ ID NO: 3 - DNA, 21 bp, Artificial Sequence
  Sense strand siRNA.  Synthetic oligonucleotides used in inhibitory RNA (RNAi) suppression of mda-9/syntenin gene
cuugaagguagacaaaguatt

SEQ ID NO: 4 - DNA, 21 bp, Artificial Sequence
  Antisense strand siRNA.  Synthetic oligonucleotides used in inhibitory RNA (RNAi) suppression of mda-9/syntenin gene
uacuuugucuaccuucaagtt

SEQ ID NO: 5 - DNA, 18 bp, Artificial Sequence
  Synthetic oligonucleotides used in RaSH procedure, all contain an XhoI restriction enzyme site. XDNP-18
ctgatcactcgagagatc

SEQ ID NO: 6 - DNA, 14 bp, Artificial Sequence
  Synthetic oligonucleotides used in RaSH procedure, all contain an XhoI restriction enzyme site. XDPN-14
ctgatcactcgaga

SEQ ID NO: 7 - DNA, 12 bp, Artificial Sequence
  Synthetic oligonucleotides used in RaSH procedure, all contain an XhoI restriction enzyme site. XDPN-12
gatctctcgagt